(19) United States
(12) Patent Application Publication
(10) Pub. No.: US 2020/0288761 A1
(43) Pub. Date: Sep. 17, 2020

(54) BISACURONE EXTRACTION METHOD

(71) Applicants: HOUSE FOODS GROUP INC., Higashiosaka-shi, Osaka (JP); HOUSE WELLNESS FOODS CORPORATION, Itami-shi, Hyogo (JP)

(72) Inventors: Muneaki TOMOTAKE, Itami-shi, Hyogo (JP); Kaori KIDA, Itami-shi, Hyogo (JP); Hiroshi SASAKO, Higashiosaka-shi, Osaka (JP)

(73) Assignees: HOUSE FOODS GROUP INC., Higashiosaka-shi, Osaka (JP); HOUSE WELLNESS FOODS CORPORATION, Itami-shi, Hyogo (JP)

(21) Appl. No.: 16/760,079

(22) PCT Filed: Oct. 31, 2017
(86) PCT No.: PCT/JP2017/039261

§ 371 Date: Apr. 29, 2020

(51) Int. Cl. A23L 33/105; C07C 49/242; A23L 2/52; A23L 5/20; A61K 31/12; A61K 36/9066

(52) U.S. Cl. CPC A23L 33/105; C07C 49/242; A23L 2/52; A23V 2002/00; A61K 31/12; A61K 36/9066; A23L 5/23

(57) ABSTRACT

It is an object of the present invention to provide a novel means for purifying bisacurone from turmeric at low costs and in a high concentration.

A method of producing a bisacurone-containing turmeric extract, comprising a stepwise extraction step of using a plurality of extraction solvents each having different polarity by combining them with one another in a predetermined order.

### TECHNICAL FIELD

The present invention relates to an extraction method capable of purifying bisacurone in a high concentration from turmeric without performing column chromatography purifications, and a bisacurone-containing turmeric extract obtained by using the method.

### BACKGROUND ART

Bisacurone is a trace component contained in turmeric, and it has been known that that bisacurone has an activity to suppress hangover symptoms occurring after alcohol ingestion, as with curcumin (a yellow pigment) that is also a component contained in turmeric (Patent Literature 1). Patent Literature 1 discloses that ingestion of bisacurone in a single oral intake of 0.5 mg or more is necessary to obtain the effect of the bisacurone to suppress hangover symptoms.

Patent Literature 2 discloses that bisacurone was extracted from turmeric according to water extraction, and that the amount of bisacurone in the obtained turmeric extract was approximately 0.15% by mass. Since this turmeric extract contains many components other than bisacurone, there might be a case where such other components had a serious influence on the taste of foods and beverages, into which the turmeric extract was mixed. In order to eliminate the influence of other components on the taste, the concentration (purity) of bisacurone in the turmeric extract is required to be higher.

As such high-concentration bisacurone, bisacurone having a purity of 99.5% or more is commercially available as a standard reagent. This bisacurone product is prepared through a step of separating and purifying bisacurone by using a column chromatography. However, high costs are required to perform such a column chromatography. Thus, it is considered practically impossible to use such high-concentration bisacurone, which is prepared by the aforementioned methods, as a raw material for foods and beverages

Under such circumstances, it has been strongly desired to develop a novel means for purifying bisacurone in a high concentration at low costs (i.e., without performing column chromatography purifications).

### CITATION LIST

### Patent Literature

Patent Literature 1: Japanese Patent No. 5543656

Patent Literature 2: Japanese Patent No. 5595614

### SUMMARY OF INVENTION

### Technical Problem

It is an object of the present invention to provide a novel means for purifying bisacurone in a high concentration from turmeric at low costs (i.e., without performing column chromatography purifications).

### Solution to Problem

As a result of intensive studies conducted towards achieving the aforementioned object, the present inventors have found that bisacurone can be dissolved in either water with high polarity or an organic solvent with low polarity, arising from a change in the intramolecular structure thereof. The present inventors have found that, by utilizing these properties of bisacurone and using a plurality of extraction solvents each having different polarity by combining them with one another in a predetermined order, bisacurone can be purified in a high concentration from turmeric without performing column chromatography purifications. The present invention has been made based on these findings, and it includes the following embodiments.
[1] A method of producing a bisacurone-containing turmeric extract, comprising:
(1) a step of adding and mixing an aqueous solvent into turmeric, extracting a water-soluble component from the turmeric, followed by solid-liquid separation, and then recovering a solution comprising the water-soluble component;
(2) a step of adding and mixing a hydrophobic organic solvent into the solution obtained in the step (1), and then separating and recovering a hydrophobic organic solvent phase;
(3) a step of removing the hydrophobic organic solvent from the obtained hydrophobic organic solvent phase to obtain a dried solid matter; and
(4) a step of adding and mixing an aqueous solvent into the obtained dried solid matter, followed by solid-liquid separation, and then recovering a solution.
[2] The method according to the above [1], wherein the solubility parameter (SP value) of the hydrophobic organic solvent is less than 9.5.
[3] The method according to the above [1] or [2], wherein the hydrophobic organic solvent is ethyl acetate, chloroform, or butyl acetate.
[4] A method of producing a bisacurone-containing turmeric extract, comprising:
(1) a step of adding and mixing a mixed solvent of a hydrophilic organic solvent and water into turmeric, extracting a water-soluble component from the turmeric, followed by solid-liquid separation, and then recovering a solution comprising the water-soluble component;
(2) a step of removing the solvent from the solution obtained in the step (1) to obtain a dried solid matter; and
(3) a step of adding and mixing water into the obtained dried solid natter, followed by solid-liquid separation, and then recovering an aqueous solution.
[5] The method according to the above [4], wherein the mixed solvent consists of acetone, ethanol, acetonitrile or methanol, and water.
[6] A bisacurone-containing turmeric extract produced by the method according to any one of the above [1] to [5].
[7] A food and beverage composition comprising the bisacurone-containing turmeric extract according to the above [6].

### Advantageous Effects of Invention

According to the present invention, bisacurone can be purified in a high concentration from turmeric without performing column chromatography purifications.

### BRIEF DESCRIPTION OF DRAWINGS

FIG. 1 is a chart diagram showing the results of HPLC analysis performed on a bisacurone-containing turmeric extract obtained using a hydrophilic organic solvent.

FIG. 2 is a chart diagram showing the results of HPLC analysis performed on a bisacurone-containing turmeric extract obtained using a hydrophobic organic solvent.

FIG. 3 is a chart diagram showing the results of HPLC analysis performed on a water extract of turmeric.

FIG. 4 is a chart diagram showing the results of HPLC analysis performed on a bisacurone-containing turmeric extract after column chromatography purification.

### DESCRIPTION OF EMBODIMENTS

In the present invention, “bisacurone” means a compound classified into bisaborane-type sesquiterpenes, which has the following planar structural formula, or a salt thereof. Bisacurone has asymmetric carbons at positions indicated with the symbol * in the planar structural formula, and thus, this compound includes several optical isomers. The term “bisacurone” used in the present description means any one of such optical isomers. [Formula 1]

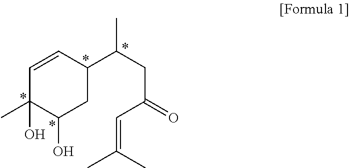 [SMILES: CC(C)=CC(=O)CC(C)C1C=CC(C)(O)C(O)C1]

In the present invention, the term “turmeric” includes plants belonging to Zingiberaceae, genus Curcuma. Thus, the turmeric used in the present invention is not particularly limited, as long as it contains bisacurone. Examples of the “turmeric” that can be utilized in the present invention may include Curcuma longa (alias: autumn turmeric), Curcuma aromatica (alias: spring turmeric), Curcuma zedoaria (alias: gadget or purple turmeric), Curcuma phaeocaulis, Curcuma kwangsiensis, Curcuma wenyujin, and Curcuma xanthorrhiza. Curcuma longa is especially preferable.

Turmeric may contain any part containing bisacurone (for example, rhizome) of turmeric. The shape of the part of turmeric is not particularly limited, as long as it is suitable for extraction of bisacurone. Thus, turmeric, which is in the original shape, or in a shape cut into an appropriate size or shape, or in a crushed shape, can be used. The turmeric having these shapes may also be dried, as appropriate. The cutting or crushing of turmeric can be carried out according to a known method. The cutting or crushing can be carried out by using an apparatus such as a mill, a mixer, or a homogenizer.

The method of the present invention includes: an embodiment utilizing a hydrophobic organic solvent in the extraction and/or purification step (a first embodiment): and an embodiment not utilizing a hydrophobic organic solvent (but utilizing a hydrophilic organic solvent) in the extraction and/or purification step (a second embodiment).

Hereinafter, individual embodiments will be described.

### [1. First Embodiment of Utilizing Hydrophobic Organic Solvent]

The method of the present invention comprises the following steps:
(1) a step of extracting a water-soluble component from turmeric and then recovering a solution comprising the water-soluble component;
(2) a step of adding and mixing a hydrophobic organic solvent into the obtained solution and then separating and recovering a hydrophobic organic solvent phase;
(3) a step of removing the hydrophobic organic solvent from the obtained hydrophobic organic solvent phase to obtain a dried solid matter; and
(4) a step of recovering the water-soluble component from the obtained dried solid matter.

Hereafter, individual steps will be described.

(1) Step of Extracting a Water-Soluble Component from Turmeric and then Recovering a Solution Comprising the Water-Soluble Component

In the present step, a water-soluble component containing bisacurone in turmeric is extracted and recovered using an extraction solvent.

Extraction of a water-soluble component from turmeric can be carried out by mixing the above-described turmeric with a solvent for extracting a water-soluble component. The extraction solvent is not particularly limited, as long as it is an aqueous solvent that can be used to extract a water-soluble component from turmeric and is not miscible with a hydrophobic organic solvent as described below. Examples of such an aqueous solvent may include water, hot water, and a mixed solvent comprising such water or hot water and a lower alcohol hydrophilic organic solvent (e.g., ethanol, methanol, or isopropanol).

The mixing of turmeric with an aqueous solvent can be carried out by immersing the turmeric in an extraction solvent at 5° C. to 100° C. for 3 minutes or more (e.g., 1 hour to 72 hours, and preferably 18 hours to 72 hours). Shaking or stirring may be added, as necessary. The shaking or stirring method can be determined by a practitioner, as appropriate.

The amount of an aqueous solvent added to turmeric is, for example, 1 to 500 parts by weight, preferably 1 to 100 parts by weight, and more preferably 5 to 50 parts by weight, based on 1 part by weight of the turmeric. If the amount of the extraction solvent added is too small, there may be a case where a water-soluble component cannot be sufficiently extracted. In contrast, if the amount of the extraction solvent added is too large, there may be a case where unfavorable problems would occur, such as a reduction in work efficiency in the subsequent operations.

Subsequently, the obtained extract is subjected to solid-liquid separation to recover a liquid portion, so that water-insoluble components can be removed and at the same time, a solution containing a water-soluble component of interest can be obtained. Solid-liquid separation can be carried out according to a common method such as centrifugation or filtration. Centrifugation can be carried out, for example, by adding centrifugal force of 1000 G or more. Filtration can be carried out, for example, using a filter with a size of 0.45 μm or less (applicable methods are not limited thereto).

The obtained solution can be concentrated, as necessary. Concentration of the solution can be carried out according to a common method such as distillation.

To the obtained solution, an aqueous solvent may be added again, as necessary, so that the present step, namely, extraction with an aqueous solvent may be carried out repeatedly.

(2) Step of Adding and Mixing a Hydrophobic Organic Solvent into the Obtained Solution and then Separating and Recovering a Hydrophobic Organic Solvent Phase

In the present step, a hydrophobic organic solvent is added and mixed into the solution comprising a water-soluble component obtained in the step (1), so that bisacurone in the solution obtained in the step (1) is transferred into the hydrophobic organic solvent.

The hydrophobic organic solvent is not particularly limited, as long as bisacurone can be transferred from the solution obtained in the step (1) into the hydrophobic organic solvent. For example, a hydrophobic organic solvent, in which a solubility parameter (hereinafter referred to as an “SP value”) is less than 9.5, and preferably in the range of 8.5 to 9.5, can be utilized. Examples of such a hydrophobic organic solvent may include chloroform (9.4), ethyl acetate (9.0), and butyl acetate (8.5), but are not limited thereto (the value in the parentheses indicates the SP value).

The amount of the hydrophobic organic solvent added into the solution obtained in the step (1) is, for example, 0.5 to 10 parts by weight, and preferably 1 to 3 parts by weight, based on 1 part by weight of the solution obtained in the step (1). If the amount of the hydrophobic organic solvent added is too small, there may be a case where bisacurone cannot be sufficiently transferred. In contrast, if the amount of the hydrophobic organic solvent added is too large, there may be a case where unfavorable problems would occur, such as a reduction in work efficiency in the subsequent operations.

The mixing of the solution obtained in the step (1) with the hydrophobic organic solvent can be carried out, for example, by shaking or stirring the mixture for 60 seconds to 3600 seconds. The shaking or stirring method can be determined by a practitioner, as appropriate.

Subsequently, the hydrophobic organic solvent phase and the aqueous solvent phase are subjected to phase separation, and thereafter, the aqueous solvent phase is removed or the hydrophobic organic solvent phase is recovered, so that unnecessary water-soluble components can be removed and the hydrophobic organic solvent phase, into which bisacurone is transferred, can be obtained. The phase separation between the hydrophobic organic solvent phase and the aqueous solvent phase can be carried out by a common method. Phase separation methods such as centrifugation, or leaving at rest can be applied.

(3) Step of Removing the Hydrophobic Organic Solvent from the Obtained Hydrophobic Organic Solvent Phase to Obtain a Dried Solid Matter

In the present step, the hydrophobic organic solvent is removed, and a component containing bisacurone, which is comprised in the solvent, is recovered.

The removal of the hydrophobic organic solvent can be carried out by a common method. Vacuum drying, ventilation drying, heat drying, air drying, freeze-drying or other drying methods can be selected, as appropriate, depending on the hydrophobic organic solvent to be removed.

(4) Step of Recovering the Water-Soluble Component from the Obtained Dried Solid Matter

In the present step, an aqueous solvent is added and mixed into the obtained dried solid matter, so that bisacurone is dissolved in the aqueous solvent and is recovered.

The aqueous solvent is not particularly limited, as long as bisacurone contained in the dried solid matter can be dissolved therein and water-insoluble components contained in the dried solid matter cannot be dissolved therein. Examples of such an aqueous solvent may include water or hot water, lower alcohol (e.g., ethanol, methanol, isopropanol), and both of them, but examples thereof are not limited thereto. When both such water and a lower alcohol hydrophilic organic solvent are used, they may be successively added, or may be added simultaneously (or as a mixed solvent).

The amount of the aqueous solvent added to the dried solid matter is, for example, 1 to 10 parts by weight, and preferably 2 to 4 parts by weight, based on 1 part by weight of the dried solid matter. If the amount of the aqueous solvent added is too small, there may be a case where bisacurone cannot be sufficiently dissolved therein. In contrast, if the amount of the aqueous solvent added is too large, there may be a case where unfavorable problems would occur, such as a reduction in work efficiency in the subsequent operations.

The mixing of the dried solid matter with the aqueous solvent may be carried out to such an extent that the dried solid matter is disintegrated. The mixing of the dried solid matter with the aqueous solvent can be carried out, for example, by shaking or stirring the mixture for 60 seconds to 3600 seconds. The shaking or stirring method can be determined by a practitioner, as appropriate.

Subsequently, the obtained extract is subjected to solid-liquid separation to recover a liquid portion, so that water-insoluble components can be removed and at the same time, a solution that contains bisacurone of interest in an aqueous solvent can be obtained. Solid-liquid separation can be carried out by a common method, such as centrifugation or filtration mentioned above.

The obtained solution containing bisacurone can be directly used as a “bisacurone-containing turmeric extract,” but it may be further dried. The obtained solution is dried to a water content of, for example, 15% by weight or less, and preferably 8% to 12% by weight. Moreover, the dried matter may be crushed, as appropriate.

Drying can be carried out by a common method such as hot-air drying, spray drying or freeze-drying.

Examples of the crushing method applied in the crushing may include a stamp mill, a mass colloider, comitrol, and a wooden pestle. The granularity of a crushed product can be adjusted using a sieve, as necessary.

According to the method of the present invention, bisacurone can be extracted and/or purified from turmeric without performing column chromatography purification, at a high purity (for example, 0.5% or more, 1% or more, 5% or more, 10% or more, 15% or more, 20% or more, or 25% or more) and also at a high recovery rate (for example, 50% or more, 60% or more, 65% or more, or 70% or more).

In one embodiment, the method of the present invention comprises:
(1) a step of adding water to turmeric to extract a water-soluble component from the turmeric, and then removing water-insoluble components to recover an aqueous solution containing the water-soluble component;
(2) a step of adding and mixing ethyl acetate or chloroform into the aqueous solution obtained in the step (1) for phase separation, and then removing a water phase and recovering an organic solvent phase,
(3) a step of removing the organic solvent from the obtained organic solvent phase according to vacuum distillation to obtain a dried solid matter; and
(4) a step of adding lower alcohol to the obtained dried solid matter to dissolve the dried solid matter therein, then further adding water thereto, and removing water-insoluble components to recover a solution containing a water-soluble component.

According to this embodiment, bisacurone can be extracted and/or purified at a high purity and a high recovery rate.

### [2. Second Embodiment of Utilizing Hydrophilic Organic Solvent]

The method of the present invention comprises the following steps:
(1) a step of adding and mixing a mixed solvent of a hydrophilic organic solvent and water into turmeric, extracting a water-soluble component from the turmeric, and then recovering a solution comprising the water-soluble component;
(2) a step of removing the solvent from the obtained solution to obtain a dried solid matter; and
(3) a step of adding and mixing water into the obtained dried solid matter, and recovering the water-soluble component.

Hereafter, individual steps will be described.

(1) Step of Adding and Mixing a Mixed Solvent of a Hydrophilic Organic Solvent and Water Into Turmeric, Extracting a Water-Soluble Component from the Turmeric, and then Recovering a Solution Comprising the Water-Soluble Component

In the present step, a water-soluble component containing bisacurone is extracted and/or recovered from turmeric, using a mixed solvent of a hydrophilic organic solvent and water (hereinafter simply referred to as a “mixed solvent”).

The hydrophilic organic solvent is not particularly limited, as long as it enables extraction of a water-soluble component from turmeric, when it is used by being mixed with water. For example, a hydrophilic organic solvent having an SP value of less than 15, and preferably in the range of 10 to 15, can be used. Examples of such a hydrophilic organic solvent may include acetone (10.0), ethanol (12.7), methanol (14.5), isopropanol (11.5), and acetonitrile (11.9), but are not limited thereto (the value in the parentheses indicates an SP value).

The mixing ratio between the hydrophilic organic solvent and water is not particularly limited, and the hydrophilic organic solvent can be preferably comprised at a weight percentage of 90% or less and 30% or more. For example, when acetone is used as a hydrophilic organic solvent, the acetone can be mixed with water, such that the weight ratio between the acetone and the water is 90:10 to 30:70, and is preferably 80:20 to 30:70. On the other hand, when ethanol is used as a hydrophilic organic solvent, the ethanol can be mixed with water, such that the weight ratio between the ethanol and the water is 90:10 to 30:70, and is preferably 80:20 to 30:70. On the other hand, when methanol is used as a hydrophilic organic solvent, the methanol can be mixed with water, such that the weight ratio between the methanol and the water is 90:10 to 30:70, and is preferably 80:20 to 30:70.

By using the hydrophilic organic solvent and water at the above-described mixing ratio, bisacurone can be extracted and/or purified at a high purity and/or at a high recovery rate.

Extraction of a water-soluble component from turmeric can be carried out by mixing the above-described turmeric with the mixed solvent. The mixing of the turmeric with the mixed solvent can be carried out by immersing the turmeric in an extraction solvent at 5° C. to 60° C. for 3 minutes or more (e.g., 1 hour to 72 hours, and preferably 18 hours to 72 hours). Shaking or stirring may be added, as necessary. The shaking or stirring method can be determined by a practitioner, as appropriate.

The amount of the mixed solvent added to the turmeric is, for example, 1 to 500 parts by weight, preferably 1 to 100 parts by weight, and more preferably 5 to 50 parts by weight, based on 1 part by weight of the turmeric. If the amount of the extraction solvent added is too small, there may be a case where a water-soluble component cannot be sufficiently extracted. In contrast, if the amount of the extraction solvent added is too large, there may be a case where unfavorable problems would occur, such as a reduction in work efficiency in the subsequent operations.

Subsequently, the obtained extract is subjected to solid-liquid separation to recover a liquid portion, so that water-insoluble components can be removed and at the same time, a solution containing the water-soluble component of interest can be obtained. Solid-liquid separation can be carried out by a common method, such as centrifugation or filtration mentioned above.

To the obtained solution, a mixed solvent may be added again, as necessary, so that the present step, namely, extraction with a mixed solvent may be carried out repeatedly.

(2) Step of Removing the Solvent from the Obtained Solution to Obtain a Dried Solid Matter

In the present step, the solvent (mixed solvent) is removed from the solution obtained in the step (1), and a component containing bisacurone contained in the solvent is then recovered.

The removal of the solvent can be carried out by a common method. Vacuum drying, ventilation drying, heat drying, air drying, freeze-drying, or other drying methods can be selected, as appropriate, depending on the solvent to be removed. For example, non-reactive gas (e.g., nitrogen gas, argon gas, or helium gas) is sprayed to the solution obtained in the step (1), so that the solution can be evaporated and/or dried and the solvent can be removed to obtain a dried solid matter of interest.

### (3) Step of Adding and Mixing Water Into the Obtained Dried Solid Matter, and Recovering the Water-Soluble Component

In the present step, water is added and mixed into the obtained dried solid matter, so that bisacurone is dissolved in the water and is recovered.

The amount of water added to the dried solid matter is, for example, 0.5 to 10 parts by weight, and preferably 1 to 3 parts by weight, based on 1 part by weight of the dried solid matter. If the amount of the water added is too small, there may be a case where bisacurone cannot be sufficiently dissolved therein. In contrast, if the amount of the water added is too large, there may be a case where unfavorable problems would occur, such as a reduction in work efficiency in the subsequent operations.

The mixing of the dried solid matter with water may be carried out to such an extent that the dried solid matter is disintegrated. The mixing of the dried solid matter with water can be carried out, for example, by shaking or stirring the mixture for 60 seconds to 3600 seconds. The shaking or stirring method can be determined by a practitioner, as appropriate.

Subsequently, the obtained extract is subjected to solid-liquid separation to recover a liquid portion, so that water-insoluble components can be removed and at the same time, an aqueous solution containing bisacurone of interest can be obtained. Solid-liquid separation can be carried out by a common method, such as centrifugation or filtration mentioned above.

The obtained aqueous solution containing bisacurone can be directly used as a “bisacurone-containing turmeric extract.” However, the aqueous solution containing bisacurone may be further dried, and for example, it may be dried to a water content of 15% by weight or less, and preferably to 8% to 12% by weight. Moreover, the dried product may be crushed, as appropriate.

The drying method and the crushing method used in crushing may be the aforementioned methods.

According to the method of the present invention, bisacurone can be extracted and/or purified from turmeric at a high purity (for example, 0.5% or more, 1.0% or more, 1.5% or more, 2.0% or more, or 2.5% or more) and also at a high recovery rate (for example, 50% or more, 60% or more, 65% or more, 70% or more, 75% or more, 80% or more, or 85% or more).

In one embodiment, the method of the present invention comprises:
(1) a step of adding and mixing a mixed solvent comprising acetone, ethanol or methanol and water to turmeric to extract a water-soluble component from the turmeric, and then removing water-insoluble components to recover a solution containing the water-soluble component;
(2) a step of evaporating and removing the solvent from the obtained solution to obtain a dried solid matter; and
(3) a step of adding water to the obtained dried solid matter to dissolve the dried solid matter therein, and removing water-insoluble components to recover an aqueous solution containing a water-soluble component.

According to this embodiment, bisacurone can be extracted and/or purified from the turmeric at a high purity and a high recovery rate.

The bisacurone-containing turmeric extracts obtained by the methods of the above-described first embodiment and second embodiment can be utilized by being added to various foods and beverages or pharmaceutical products, without being limited to specific use aspects.

Specifically, the bisacurone-containing turmeric extract of the present invention is combined, as necessary, with other materials that are acceptable as foods and beverages, and can be used in the form of a food and beverage composition. Since bisacurone has an activity to alleviate hangover symptoms after alcohol ingestion (Japanese Patent No. 5543656), the present food and beverage composition may have an indication such as, for example, an indication regarding the activity to alleviate hangover symptoms, on the product body, packaging, instructions, advertisement, or promotional electronic information thereof. The shape of the food and beverage composition is not particularly limited, and it may have various shapes such as a solid, a semi-solid, or a liquid.

Moreover, the bisacurone-containing turmeric extract of the present invention can be formulated by being combined with a pharmaceutically acceptable carrier, additive, excipient or other components, as necessary, and can be used in the form of a pharmaceutical composition. The present pharmaceutical composition can be utilized to treat or prevent hangover symptoms. The administration form of the present pharmaceutical composition is not particularly limited, and it is appropriately selected, as necessary. In general, the present pharmaceutical composition can be administered as an oral agent such as a tablet, a capsule, a granule, a fine grain agent, a powder agent, a liquid agent, a syrup agent, a suspending agent, an emulsion or an elixir, or a parenteral agent such as an injection, an infusion, a suppository, an inhalant, a transdermal absorbent, a transmucosal absorbent, a patch or an ointment. The carrier, additive, excipient or other components used in the present pharmaceutical composition can be selected, as appropriate, depending on the administration route.

Hereinafter, the present invention will be more specifically described in the following examples. However, the following examples are not intended to limit the technical scope of the present invention.

### EXAMPLES

### [Production of Standard Samples]

10 mg of Bisacurone (manufactured by Nagara Science Co., Ltd.) was precisely weighed into a 500-mL beaker, and 500 mL of a 30% acetonitrile (Wako Pure Chemical Industries, Ltd.) solution was then added into the beaker, so that bisacurone was dissolved in the solution to obtain a 20 ppm bisacurone standard sample. The obtained standard sample was further diluted with a 30% acetonitrile solution, to prepare 10 ppm, 8 ppm, 4 ppm, 2 ppm, and 0.8 ppm standard samples.

### [Production of Turmeric Crushed Product]

Appropriate amounts of turmeric chips obtained by drying the rhizome of autumn turmeric (Curcuma longa) were crushed with a coffee mill IFM-800 (manufactured by IWATANI) for 2 minutes to prepare a turmeric crushed product.

### [Pre-Treatment Performed Before HPLC Measurement]

Each final sample was diluted with a 30% acetonitrile solution to result in a bisacurone concentration of 20 ppm or less, and was then filtrated through a 0.45 μm filter to prepare a sample solution.

### [HPLC Measurement Conditions]

Apparatus: Waters ACQUITY H-Class System

Column: Waters XBridge C18, 5 μm, 6×250 mm

Temperature: 50° C.

Flow rate: 1.0 mL/min

Mobile phase: 30% acetonitrile (pH 3.3) TFA water (Wako Pure Chemical Industries, Ltd.)

Detector: UV 240 nm

### (Test 1) Extraction Method Utilizing Hydrophilic Organic Solvent

In the present test, three types of hydrophilic organic solvents, namely, acetone, ethanol and methanol were used.

An appropriate amount of turmeric crushed product was weighed into a 1.5 mL Eppendorf tube, and it was defined to be an initial weight. 1 mL of each hydrophilic organic solvent in a predetermined concentration was added into this turmeric crushed product, and then, the tube was capped and hermetically sealed with Parafilm. Thereafter, shaking and stirring was performed using Vortex for 16 hours.

Subsequently, the reaction mixture was centrifuged at 15000 rpm for 60 minutes for solid-liquid separation.

The supernatant was recovered, and nitrogen gas was then sprayed on the surface thereof to evaporate the hydrophilic organic solvent. Then, distilled water was added thereto to a total amount of 1.5 mL, so that hydrophobic components were precipitated.

Subsequently, the resultant was centrifuged at 15000 rpm for 60 minutes for solid-liquid separation, and the supernatant was recovered and freeze-dried (at −50° C. for 72 hours) for powderization. The weight of the thus obtained powders was defined to be a weight after drying.

Thereafter, an appropriate amount of a 30% acetonitrile (Wako Pure Chemical Industries, Ltd.) solution was added to the powders, so that the powders were diluted to result in a bisacurone concentration of 20 ppm or less upon HPLC measurement, followed by performing the HPLC measurement. The bisacurone concentration at this time was defined to be a concentration upon measurement.

Moreover, the final bisacurone amount was calculated from the concentration upon measurement, and the bisacurone concentration in the dried powders was then calculated using the weight after drying. The obtained value was defined to be purity (%).

Furthermore, the bisacurone concentration in the used turmeric crushed product was obtained by extracting bisacurone from the turmeric crushed product by shaking and stirring the product using 50% ethanol (Wako Pure Chemical Industries, Ltd.) for 16 hours, and performing the HPLC measurement. As a result, the bisacurone concentration was calculated to be 0.59 mg/g. The amount of bisacurone (theoretical value) in the initial turmeric crushed product was calculated from the above-obtained value and the initial weight, and the actually recovered amount was compared with the theoretical value to obtain a recovery rate (%).

Besides, in a comparative example, an extract obtained by using water instead of the above-described hydrophilic organic solvent and performing the same treatment as described above was used.

The results are shown in Table 1.

[Table 1]

TABLE 1

| | | | Weight | Concentration | | |
|---|---|---|---|---|---|---|
| | | Initial | after | upon | | |
| | Extraction | weight | drying | measurement | Purity | Recovery |
| | solvent | (mg) | (mg) | (ppm) | (%) | rate (%) |
| Example 1 | Acetone 100% | 35.3 | 0.17 | 4.10 | 2.41% | 19.68% |
| Example 2 | Acetone 90% | 28.5 | 0.16 | 4.01 | 2.50% | 23.83% |
| Example 3 | Acetone 80% | 23 | 0.44 | 10.96 | 2.49% | 80.76% |
| Example 4 | Acetone 50% | 22.5 | 1.67 | 9.94 | 0.60% | 74.90% |
| Example 5 | Acetone 30% | 33.4 | 2.7 | 17.02 | 0.63% | 86.36% |
| Example 6 | Ethanol 100% | 41.1 | 0.09 | 1.68 | 1.86% | 6.91% |
| Example 7 | Ethanol 90% | 17 | 0.1 | 2.09 | 2.09% | 20.83% |
| Example 8 | Ethanol 80% | 36.3 | 0.81 | 15.50 | 1.91% | 72.39% |
| Example 9 | Ethanol 50% | 25.9 | 0.63 | 11.35 | 1.80% | 74.30% |
| Example 10 | Ethanol 30% | 41.4 | 1.86 | 19.37 | 1.04% | 79.30% |
| Example 11 | Methanol 100% | 35.2 | 0.25 | 4.23 | 1.69% | 20.35% |
| Example 12 | Methanol 90% | 47.1 | 0.45 | 5.60 | 1.24% | 20.15% |
| Example 13 | Methanol 80% | 28.7 | 1.12 | 8.95 | 0.80% | 52.87% |
| Example 14 | Methanol 50% | 43.7 | 2.25 | 16.38 | 0.73% | 63.55% |
| Example 15 | Methanol 30% | 21.3 | 1.05 | 7.70 | 0.73% | 61.28% |
| Comparative | Water 100% | 48.5 | 8.4 | 22.99 | 0.15% | 44.03% |
| Example | | | | | | |

From the above results, it was found that according to the present extraction method, a turmeric extract containing bisacurone could be obtained at a significantly high purity and at a significantly high recovery rate, in comparison to the conventional method of obtaining bisacurone from turmeric according to water extraction taught by Japanese Patent No. 5595614.

In particular, by performing initial extraction using a relatively high concentration of hydrophilic organic solvent, a turmeric extract containing bisacurone at a high purity could be obtained. It is considered that this is because extraction of hydrophilic substances other than bisacurone can be suppressed in the initial extraction, and also because hydrophobic substances other than bisacurone can be precipitated and/or removed in the final addition of water.

On the other hand, as the concentration of the hydrophilic organic solvent approached to 100%, a significant reduction in the recovery rate of bisacurone was observed. From these results, it was confirmed that, in order to efficiently extract bisacurone from turmeric, a hydrophilic organic solvent and a certain amount of water are necessary as extraction solvents used in the initial extraction.

As described above, it was revealed that a bisacurone-containing turmeric extract having a high purity and a high recovery rate can be efficiently obtained by performing an initial extraction using a 30% to 80% hydrophilic organic solvent, and also by precipitating and/or removing hydrophobic substances by final addition of water.

### (Test 2) Extraction Method Utilizing Hydrophobic Organic Solvent

In the present test, two types of hydrophobic organic solvents, namely, ethyl acetate and chloroform were used.

A turmeric crushed product (100 g, initial weight) was weighed into a 3 L stainless steel mug, and distilled water (2 L) was then added thereto, followed by stirring and mixing for 16 hours.

Subsequently, the reaction mixture was filtrated through a filter (No. 5B), and a filtrate was then recovered. The recovered filtrate was concentrated into a final amount of approximately 400 mL at 60° C., using an evaporator.

The obtained concentrate was transferred into a 2 L separatory funnel, and each hydrophobic organic solvent (700 mL) was then added to the separatory funnel. The obtained mixture was stirred for 2 minutes and was then left at rest for 10 minutes.

After water and the hydrophobic organic solvent had been separated into two phases, the water phase on the lower side was removed. When the bisacurone concentration in the lower water phase was measured, almost no bisacurone was detected. Thus, it was confirmed that the bisacurone was transferred into the hydrophobic organic solvent phase.

Subsequently, the hydrophobic organic solvent was removed from the recovered hydrophobic organic solvent phase, using an evaporator, and was then dried and solidified.

Subsequently, ethanol (20 mL) was added to the resultant, so that the resultant was dissolved in the ethanol. Thereafter, distilled water (800 mL) was added to the solution to precipitate hydrophobic components.

After that, the resultant was centrifuged at 12000 rpm for 60 minutes for solid-liquid separation, and the supernatant was then freeze-dried (at −50° C. for 72 hours) for powderization. The weight of the thus obtained powders was defined to be a weight after drying.

Thereafter, 500 mL of a 30% acetonitrile (Wako Pure Chemical Industries, Ltd.) solution was added to the powders, and HPLC measurement was then performed. The bisacurone concentration at this time was defined to be a concentration upon measurement.

Moreover, the final bisacurone amount was calculated from the concentration upon measurement, and the bisacurone concentration in the dried powders was then calculated using the weight after drying. The obtained value was defined to be purity (%).

Furthermore, the bisacurone concentration in the used turmeric crushed product was obtained by extracting bisacurone from the turmeric crushed product by shaking and stirring the product using 50% ethanol (Wako Pure Chemical Industries, Ltd.) for 16 hours, and then performing the HPLC measurement. As a result, the bisacurone concentration was calculated to be 0.59 mg/g. The amount of bisacurone (theoretical value) in the initial turmeric crushed product was calculated from the above-obtained value and the initial weight, and the actually recovered amount was compared with the theoretical value to obtain a recovery rate (%).

The results are shown in Table 2.

TABLE 2

| | | | | Concen- | | |
|---|---|---|---|---|---|---|
| | | | | tration | | |
| | | | Weight | upon | | |
| | Extrac- | Initial | after | measure- | | Recovery |
| | tion | weight | drying | ment | Purity | rate |
| | solvent | (g) | (g) | (ppm) | (%) | (%) |
| Example | Ethyl | 100 | 0.14 | 85.09 | 30.39% | 72.11% |
| 16 | acetate | | | | | |
| Example | Chloro- | 100 | 0.15 | 84.65 | 27.55% | 70.04% |
| 17 | form | | | | | |

From the above results, it was found that according to the present extraction method, a turmeric extract containing bisacurone could be obtained at a significantly high purity and at a significantly high recovery rate, in comparison to the conventional method of obtaining bisacurone from turmeric according to water extraction taught by Japanese Patent No. 5595614.

It is considered that this is because extraction of hydrophobic substances can be suppressed and removed by initially performing water extraction, also because almost no hydrophilic substances other than bisacurone were transferred upon transferring, since the subsequently used hydrophobic organic solvent is hardly miscible with water, and further because hydrophobic substances other than bisacurone can be precipitated and/or removed by final addition of water.

### (Test 3) HPLC Analysis of Bisacurone-Containing Turmeric Extracts Obtained by Various Types of Extraction Methods

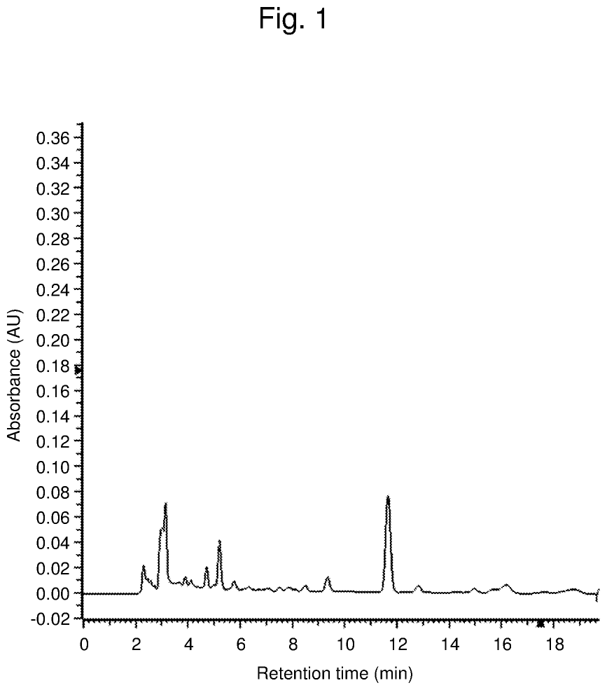

Bisacurone-containing turmeric extracts obtained by the methods of the above-described Test 1 and Test 2, a water extract of turmeric (the comparative example in Test 1), and a bisacurone-containing turmeric extract subjected to column chromatography purification (which was obtained by extracting bisacurone from the rhizome portion of autumn turmeric (Curcuma longa) with water, then re-extracting the obtained water extract with methanol, and then purifying bisacurone from the obtained methanol extract using a separatory column) were each subjected to HPLC analysis. The obtained charts are shown in FIGS. 1 to 4.

FIG. 1 shows a chart of the bisacurone-containing turmeric extract obtained by the method of Test 1.

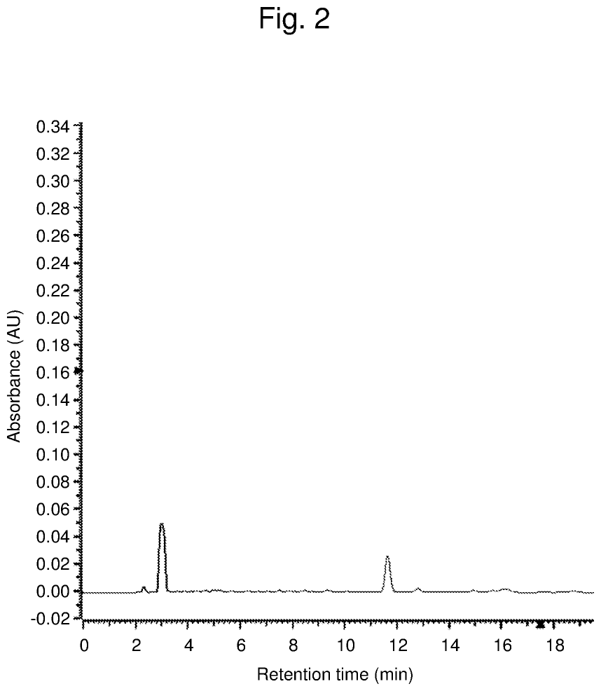

FIG. 2 shows a chart of the bisacurone-containing turmeric extract obtained by the method of Test 2.

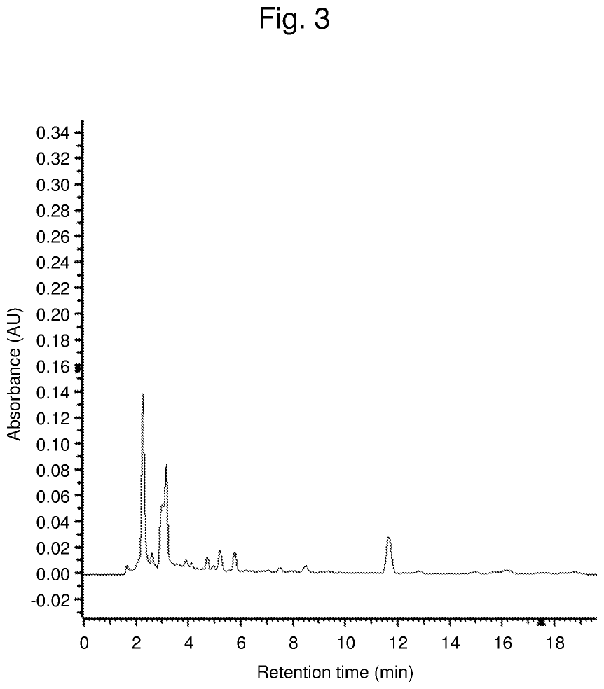

FIG. 3 shows a chart of the water extract of turmeric.

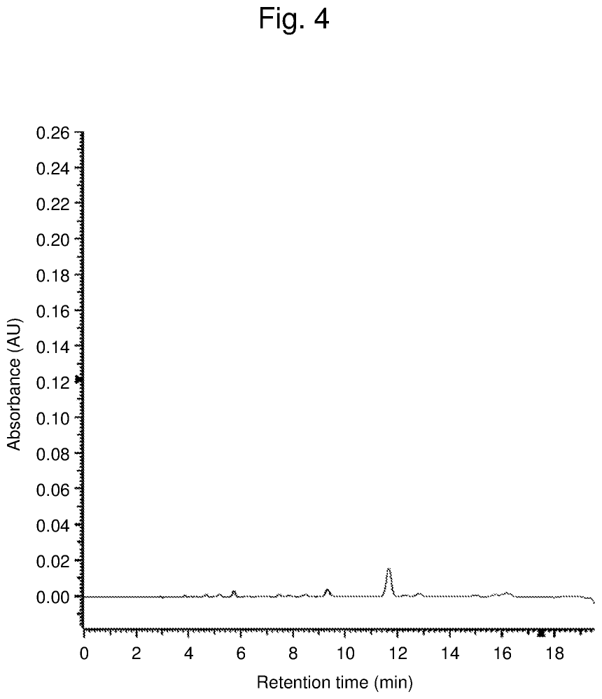

FIG. 4 shows a chart of the bisacurone-containing turmeric extract that has been subjected to column chromatography purification.

In each figure, the peak of bisacurone is shown in the retention time from 11.6 to 11.8 minutes.

It was confirmed that the amounts of components other than bisacurone in the bisacurone-containing turmeric extracts obtained by the methods of Test 1 and Test 2, which appeared in the retention time from approximately 2 minutes to 10 minutes, were reduced, in comparison to those in the water extract of numeric, although the amounts were not as small as those in the water extract of turmeric that had been subjected to column chromatography purification.

All publications, patents and patent applications cited in the present description are incorporated herein by reference in their entirety.

1. A method of producing a bisacurone-containing turmeric extract, comprising:
(1) a step of adding and mixing an aqueous solvent into turmeric, extracting a water-soluble component from the turmeric, followed by solid-liquid separation, and then recovering a solution comprising the water-soluble component;
(2) a step of adding and mixing a hydrophobic organic solvent into the solution obtained in the step (1), and then separating and recovering a hydrophobic organic solvent phase;
(3) a step of removing the hydrophobic organic solvent from the obtained hydrophobic organic solvent phase to obtain a dried solid matter; and
(4) a step of adding and mixing an aqueous solvent into the obtained dried solid matter, followed by solid-liquid separation, and then recovering a solution.

2. The method according to claim 1, wherein the solubility parameter (SP value) of the hydrophobic organic solvent is less than 9.5.

3. The method according to claim 1, wherein the hydrophobic organic solvent is ethyl acetate, chloroform, or butyl acetate.

4. A method of producing a bisacurone-containing turmeric extract, comprising:
(1) a step of adding and mixing a mixed solvent of a hydrophilic organic solvent and water into turmeric, extracting a water-soluble component from the turmeric, followed by solid-liquid separation, and then recovering a solution comprising the water-soluble component;
(2) a step of removing the solvent from the solution obtained in the step (1) to obtain a dried solid matter; and
(3) a step of adding and mixing water into the obtained dried solid matter, followed by solid-liquid separation, and then recovering an aqueous solution.

5. The method according to claim 4, wherein the mixed solvent consists of acetone, ethanol, acetonitrile or methanol, and water.

6. A bisacurone-containing turmeric extract produced by the method according to claim 1.

7. A food and beverage composition comprising the bisacurone-containing turmeric extract according to claim 6.

8. A bisacurone-containing turmeric extract produced by the method according to claim 4.

9. A food and beverage composition comprising the bisacurone-containing turmeric extract according to claim 8.
